(19) United States
(12) Patent Application Publication
(10) Pub. No.: US 2021/0299290 A1
(43) Pub. Date: Sep. 30, 2021

(54) DOWNLIGHT APPARATUS FOR UV-DEACTIVATION OF PATHOGENS

(71) Applicant: Healthe, Inc., Cocoa Beach, FL (US)

(72) Inventors: Fredric Maxik, Cocoa Beach, FL (US); Ran Zhou, Rockledge, FL (US); Addy Widjaja, Palm Bay, FL (US); Matthew Regan, Melbourne, FL (US)

(21) Appl. No.: 17/215,214

(22) Filed: Mar. 29, 2021

Related U.S. Application Data
(60) Provisional application No. 63/000,926, filed on Mar. 27, 2020.

(51) Int. Cl. A61L 2/00; F21S 8/02; F21V 23/06; F21V 23/00; F21V 23/04; A61L 2/26

(52) U.S. Cl. CPC A61L 2/0047; F21S 8/026; F21V 23/06; F21Y 2113/10; F21V 23/0471; A61L 2/26; F21V 23/003

(57) ABSTRACT

An apparatus for performing ultraviolet-deactivation of pathogens is disclosed. The disclosed apparatus includes a housing, a base member, one or more visible light assemblies, and one or more ultraviolet radiation assemblies. The housing is configured to define a downlight aperture region irradiated by the apparatus. The apparatus may additionally include sensors to detect whether a living organism occupies the downlight aperture region. The input from the sensors may be used to control the one or more ultraviolet radiation assemblies.

### RELATED APPLICATIONS

The present application claims priority to U.S. Provisional Patent Application No. 63/000,926, titled “DOWNLIGHT APPARATUS FOR UV-DEACTIVATION OF PATHOGENS,” filed Mar. 27, 2020, which is hereby incorporated by reference herein in its entirety.

### TECHNICAL FIELD

The present disclosure relates to a downlight apparatus for UV-deactivation of pathogens. The apparatus may be recessed in or installed on the ceiling so as to sanitize air surfaces below. In some embodiments, Far UV may be utilized to allow the apparatus to be safely used in the presence of humans.

### BACKGROUND

The impact of the spread of viruses has been acutely felt throughout the world in the present time. COVID-19, SARS, and other viruses and microorganisms have had a significant and deadly impact on the way that individuals live their lives. In particular, individuals are less willing and/or able to occupy public spaces, such as malls, restaurants, theaters, public transit spaces, event and conference spaces, elevators, and other crowded locations, for fear of being exposed to and succumbing to a virus.

In order to combat the spread of viruses in public spaces, various precautions have been implemented. Due to the airborne nature of many pathogens including COVID-19, covering one's face with a fabric mask and maintaining physical distance from others is recommended. Additionally, proper ventilation and filtration of air within an environment may be crucial to removing pathogens from the environment. However, standard air filtration may not efficiently remove pathogens to the degree necessary to significantly impact human-to-human transmission.

More recently, ultraviolet light has been introduced as a means to sanitize surfaces and substances. The type of ultraviolet (UV) light has been classified into at least four bands depending upon the effects upon the skin of humans and other animals. Such bands include UV-A, which is defined as ultraviolet light having a wavelength in a range from 315 nm to 400 nm; UV-B, which is defined as ultraviolet light having a wavelength in a range from 280 nm to 315 nm; UV-C, which is defined as ultraviolet light having a wavelength that is in a range from 235 nm to 280 nm; and Far UV, which is defined as ultraviolet light having a wavelength that is in a range from 185 nm to 235 nm.

Ultraviolet light in the UV-C range has been used for sanitization. For example, UV light emitted at 254 nm and 265 nm has been used to destroy viruses and other microorganisms for a number of years. Far UV light (e.g., 222 nm) has been shown to have some efficacy for this use as well. However, UV light emitted in the UV-C range can have harmful impacts on humans. For example, prolonged direct exposure to UV-C light can result in eye and skin damage, such as acute corneal injury (sometimes referred to as “welder's eye”) and acute erythema. Acute effects from UV-C light include redness, ulceration or burns of the skin. Longer-term effects may include premature aging of the skin and/or skin cancer.

Still, air sanitization is often most crucial when there is human presence. Many public spaces experience waves of high traffic (e.g., public transit stations, airport terminals, and movie theaters) and thus require robust sanitization at discrete intervals where humans are present. Permanent, continuously running UV sanitization systems may have large power requirements and may thus may be obtrusive and occupy a great deal of space, which may be unsuitable for various public spaces.

As such, it would be desirable to have a downlight sanitization system using UV light that is regulated based on the presence of humans.

### SUMMARY

This summary is provided to comply with 37 C.F.R. § 1.73, which requires a summary of the invention briefly indicating the nature and substance of the invention. It is submitted with the understanding that it will not be used to interpret or limit the scope or meaning of the present disclosure.

A downlight apparatus that utilizes UV radiation to deactivate pathogens is provided. The apparatus comprises a housing; a base member, wherein the base member is attached to the housing; a light source configured to emit light in the visible spectrum; and one or more ultraviolet radiation assemblies within the housing; wherein the housing is configured to define a downlight aperture region irradiated by the apparatus.

In some embodiments, the base member comprises a standard Edison base.

In some embodiments, the apparatus may further comprise a bracket.

In some embodiments, the downlight aperture region is configured to encompass a human.

In some embodiments, the apparatus may further comprise a control circuit coupled to the one or more ultraviolet radiation assemblies.

In some embodiments, the control circuit may additionally be coupled to one or more sensors configured to detect radiation within the downlight aperture region. The one or more sensors may be configured to detect infrared radiation from an individual occupying the aperture region. The one or more sensors may detect radiation reflected from some object in the downlight aperture region, wherein the radiation was generated by the apparatus. Alternatively, the one or more sensors may be motion detectors.

In some embodiments, data received from the one or more sensors may be used to determine whether a living being is occupying the downlight aperture region.

In some embodiments, at least one of the one or more ultraviolet assemblies may be configured to emit electromagnetic radiation having a peak intensity within a range from 200 nm to 280 nm. In further embodiments, at least one of the one or more ultraviolet radiation assemblies are configured to emit electromagnetic radiation having a peak intensity within a range from 217 nm to 227 nm or more specifically at 222 nm. In some embodiments, at least one of the one or more ultraviolet radiation assemblies are configured to emit electromagnetic radiation having a peak intensity within a range from 249 nm to 259 nm.

In some embodiments, based on a determination that a living organism is detected in the downlight aperture, the control circuit is configured to activate the light source and deactivate at least one of the one or more ultraviolet radiation assemblies.

In some embodiments, based on a determination that no living organism is detected in the downlight aperture, the control circuit is configured to deactivate the light source, activate at least one of the one or more ultraviolet radiation assemblies to irradiate the downlight aperture region, and deactivate the at least one of the one or more ultraviolet radiation assemblies after a preconfigured period of time. Activating the light source or the one or more ultraviolet radiation assemblies may be performed on preconfigured time delay.

In some embodiments, the control circuit may be configured to activate the light source based on the input from the one or more sensors indicative of a presence of one or more living organisms within the portal aperture region, deactivate the light source based on the input from the one or more sensors indicative of an absence of living organisms with the portal aperture region, and selectively activate the one or more ultraviolet radiation assemblies according to a preconfigured schedule.

In some embodiments, the one of the one or more ultraviolet radiation assemblies may include light-emitting diodes, laser diodes, a pulsed xenon laser, or a fiber laser.

In some embodiments, the apparatus may further comprise a transparent or translucent cap attached to the housing, wherein light emitted by the light source passes through the transparent or translucent cap.

### BRIEF DESCRIPTION OF THE DRAWINGS

FIG. 1 depicts a perspective view of a downlight apparatus utilizing ultraviolet (UV) radiation for the deactivation of pathogens in accordance with an embodiment.

FIG. 2 depicts a side view of the downlight apparatus utilizing ultraviolet (UV) radiation for the deactivation of pathogens in accordance with an embodiment.

FIG. 3 is an end view of the downlight apparatus utilizing ultraviolet (UV) radiation for the deactivation of pathogens in accordance with an embodiment.

### DETAILED DESCRIPTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the invention.

As used in this document, the singular forms “a,” “an,” and “the” include plural references unless the context clearly dictates otherwise. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation may be modified as would be readily apparent to a person skilled in the art. For example, in a list of numerical values such as “about 49, about 50, about 55, “about 50” means a range extending to half the interval(s) between the preceding and subsequent values, e.g., more than 49.5 to less than 52.5. Furthermore, the phrases “less than about” a value or “greater than about” a value should be understood in view of the definition of the term “about” provided herein.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” etc.). Further, the transitional term “comprising,” which is synonymous with “including,” “containing,” or “characterized by,” is inclusive or open-ended and does not exclude additional, unrecited elements or method steps. While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes most of the subject of the reference. The meaning of any of these terms is dependent upon the context within which it is used, and the meaning may be expressly modified.

As will be understood by one skilled in the art, for any and all purposes, such as in terms of providing a written description, all ranges disclosed herein are intended as encompassing each intervening value between the upper and lower limit of that range and any other stated or intervening value in that stated range. All ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells as well as the range of values greater than or equal to 1 cell and less than or equal to 3 cells. Similarly, a group having 1-5 cells refers to groups having 1, 2, 3, 4, or 5 cells, as well as the range of values greater than or equal to 1 cell and less than or equal to 5 cells, and so forth.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason. Throughout this disclosure, various patents, patent applications and publications are referenced. The disclosures of these patents, patent applications and publications are incorporated into this disclosure by reference in their entireties in order to more fully describe the state of the art as known to those skilled therein as of the date of this disclosure. This disclosure will govern in the instance that there is any inconsistency between the patents, patent applications and publications cited and this disclosure.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

It is contemplated that a radiation methodology is employed in an integrated lighting device or retrofit lighting device, such as, for example, the one shown in U.S. Pat. No. 7,824,065 (the content of which is incorporated by reference except to the extent disclosure therein is inconsistent with disclosure herein) which is configured to selectively emit UV radiation as described below. A downlight, sometimes referred to as a recessed can light or ceiling light, incudes a lamp, often a light bulb set in a cylindrical housing, that is mounted from, mounted on, or recessed into a ceiling so that a beam of light is directed downward.

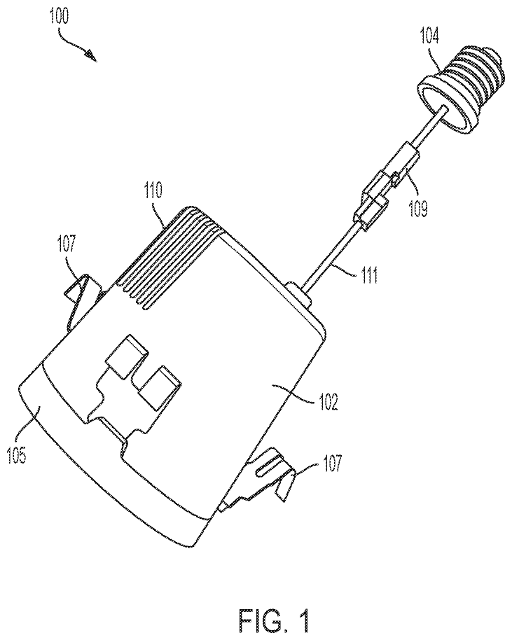

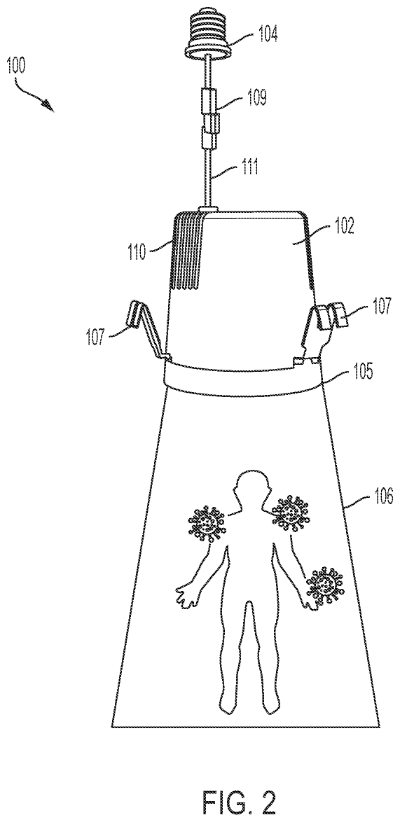

Various characteristics and features of downlight apparatuses that utilize UV radiation to deactivate pathogens are disclosed herein. Referring specifically to FIGS. 1-3, an illustrative downlight apparatus 100 is presented. The downlight apparatus 100 may comprise a housing 102 attached to a base member 104, such as a conventional Edison base, at one end (e.g. the top end) and a transparent or translucent cap 105 at the other end. The housing 102 and cap 105 may cooperate to define a downlight aperture 106. The downlight aperture 106 may be a region that is irradiated by the downlight apparatus 100 to deactivate pathogens within that space. In some embodiments, the downlight apparatus 100 may be configured to permit a human to stand within the downlight aperture 106.

In some embodiments, the housing 102 of the downlight apparatus 100 may comprise a cylinder or a canister. In some embodiments, the housing 102 includes one or more spring-loaded mounting clips 107 and a vent 110. The housing 102 may include additional and/or alternate features as will be apparent to those of ordinary skill in the art.

The downlight apparatus 100 may further comprise a radiation assembly 110 (or a plurality of radiation assemblies 110). The radiation assembly 110 may be positioned within the interior space 108 and oriented such that radiation emitted thereby may pass through the transparent or translucent cap 105 to form the downlight aperture 106.

Each radiation assembly 110 may comprise a radiation assembly housing 112 configured to be attached to and carried by the housing member 102 via one or more brackets 113. In some embodiments, a radiation-emitting device 114 is configured to emit radiation through an aperture of the radiation assembly housing 112. Control circuitry 116 may be positioned in electrical communication with the radiation-emitting device 114 and configured to provide power to and control the operation of the radiation-emitting device 114. The control circuitry 116 may be coupled to the base member 104 via an optional cable 111 and/or adapter 109.

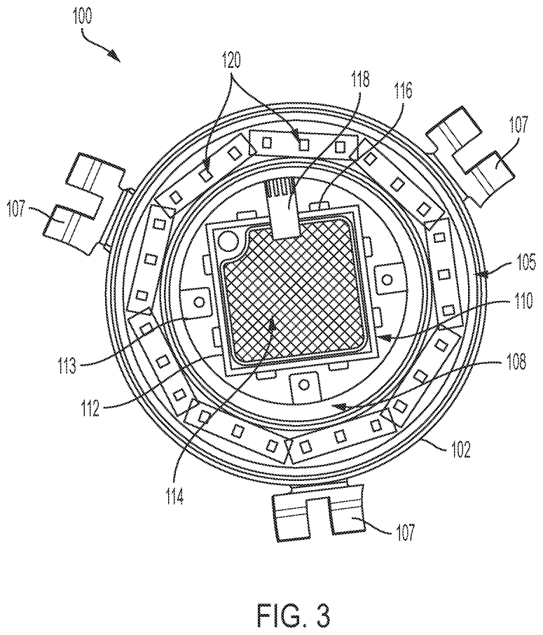

In some embodiments, the transparent or translucent cap 105 of the downlight apparatus 100 may be attached to and carried by the housing 102. The combination of the transparent or translucent cap 105 and the housing 102 may define an interior space 108 of the downlight apparatus 100. In some embodiments, the transparent or translucent cap 105 may be configured to not cover the one or more radiation assemblies 110. For example, as shown in FIG. 3, the transparent or translucent cap 105 may be ring-shaped and may extend around the radiation assembly 110. Accordingly, the transparent or translucent cap 105 may permit radiation emitted by the radiation assembly 110 to pass through the opening in the ring and into the downlight aperture 106 without diffusing through the transparent or translucent cap 105.

In some embodiments, the control circuitry 116 comprises at least one processor and any number of additional electrical components to monitor and control the function of the downlight apparatus 100. Additional components may comprise one or more non-transitory computer readable media and a wired and/or wireless communication interface.

The downlight apparatus 100 may further comprises one or more visible light assemblies 120. The visible light assemblies may comprise one or more visible light sources operably connected to the control circuitry. In some embodiments, the one or more visible light sources comprise LEDs. However, the visible light assemblies 120 may include any visible light sources as would be known to a person having an ordinary level of skill in the art. As shown in FIG. 3, the visible light sources may be covered by the transparent or translucent cap 105. Accordingly, light emitted by the one or more visible light assemblies 120 may pass through the transparent or translucent cap 105 and be diffused therethrough. Accordingly, the diffused light may be emitted to the downlight aperture 106 and the surrounding areas.

In some embodiments, each radiation assembly 110 may comprise one or more sensors 118 positioned in communication with the control circuitry 116 and operable to detect radiation either emitted by or reflected from an object within the downlight aperture 106. In some embodiments, a sensor 118 may be configured to detect infrared (IR) radiation resulting from, for example, the body heat of an individual occupying the downlight aperture 106. In some embodiments, one or more of a sensor 118 and the radiation-emitting device 114 may be operable to emit radiation that can reflect off an object occupying the downlight aperture 106 and be detected by the sensor 118 to indicate the object's presence in the downlight aperture 106. Any method and/or device for detecting objects and/or living specimens are contemplated and intended to be included within the scope of the disclosure.

In some embodiments, the control circuitry 116 may be configured to receive signals from the one or more sensors 118 and determine the presence or absence of objects and/or living specimens within the downlight aperture 106. In some embodiments, the control circuitry 116 may be configured to determine whether an object within the downlight aperture 106 is living or non-living. For example, the control circuitry 116 may interpret control signals from an infrared sensor 118 to determine whether the object within, for example, the downlight aperture 106 is emitting heat in a manner consistent with the body of a living specimen.

The radiation-emitting device 114 may be configured to emit radiation to deactivate pathogens within the downlight aperture 106. Such radiation may be within specific wavelength ranges and have a specific wavelength with a maximum intensity of radiation emitted by the radiation-emitting device. In some embodiments, the radiation-emitting device 114 may be configured to emit electromagnetic radiation having a peak intensity within the Far-UV range, i.e. within a range from 185 nanometers (nm) to 235 nm. In some embodiments, the radiation-emitting device 114 may be configured to emit electromagnetic radiation having a peak intensity within a wavelength range from 217 nm to 227 nm. In some embodiments, the radiation-emitting device 114 may be configured to emit electromagnetic radiation having a peak intensity at 222 nm.

In some embodiments, the one or more visible light assemblies 120 may comprise specific shifted wavelengths. For example, a blue LED pump used to create a white visible light source 120 may be shifted to 435 nm. Certain surfaces in the room may be lined or coated with a reactive component, such as TiO₂, to induce photocatalytic effects when a shifted visible light source 120 is activated. TiO₂ applied to the exterior surfaces may be sourced from or provided in the form of anatase, ilmenite, rutile, and/or other forms and additional or alternative reactive components may be utilized.

As mentioned above, the control circuitry 116 may be configured to differentiate between living and non-living specimens within the downlight aperture 106. In such embodiments, the radiation-emitting device 114 may further be configured to emit a second electromagnetic radiation having a peak intensity within the UV-C range, such as from 249 nm to 259 nm. In some embodiments, the radiation-emitting device 114 may be configured to emit a second electromagnetic radiation having a peak intensity at 254 nm. In some embodiments, the control circuitry 116 may be configured to emit the first electromagnetic radiation having a peak intensity at 222 nm when the object is determined to be a living organism, and to emit one or both of the first and second electromagnetic radiations having peak intensities at 222 nm and 254 nm, respectively, upon determining the object is not a living organism.

In some embodiments, the control circuitry 116 may be configured to activate the visible light assemblies 120 upon detecting a living specimen and/or to activate the radiation-emitting device 114 to emit the first electromagnetic radiation having peak intensity at 222 nm. The second electromagnetic radiation having peak intensity at 254 nm may not be emitted while the living specimen is detected.

In some embodiments the control circuitry 116 may be further configured to control the visible light assemblies 120 based on the detecting of a living specimen within the downlight aperture 106. In some embodiments the control of the radiation-emitting device 114 and/or the visible light assemblies 120 may be performed on a time delay. For example, when the control circuitry 116 detects no living specimen, after a first delay, the visible light assemblies 120 may be turned off and then, after a second delay, the radiation-emitting device 114 may be activated, emitting both of the first and second electromagnetic radiations having peak intensities at 222 nm and 254 nm. After a third, longer, delay the radiation-emitting device 114 may be deactivated. However, the visible light assemblies 120 and the radiation-emitting device 114 may be controlled in additional combinations. In some embodiments, after the third delay, the radiation-emitting device 114 may be controlled to cease emission of the second electromagnetic radiation at 254 nm while continuing to emit the first electromagnetic radiation at 222 nm.

In some embodiments, the control circuitry 116 may activate and deactivate the radiation-emitting device 114 selectively according to a schedule. In some embodiments, the radiation-emitting device 114 may be activated and deactivated at specific times. In some embodiments, the radiation-emitting device 114 may be activated and deactivated to achieve a threshold amount of irradiation time over a given time period. For example, the radiation-emitting device 114 may be activated for about 15 minutes per hours, about 30 minutes per hour, and the like. The threshold amount of irradiation time may also be calculated per day, per week, and the like. In some embodiments, irradiation may performed according to the schedule regardless of the presence of living organisms. In some embodiments, irradiation may be interrupted by the presence of living organisms and continued at a later time. Accordingly, the control circuitry 116 may track the total irradiation time for a given time period and selectively activate and deactivate the radiation-emitting device 114.

In some embodiments, the radiation-emitting device 114 may continuously emit the first electromagnetic radiation at 222 nm regardless of the detection of a living specimen. Accordingly, only the visible light assemblies 120 and the second electromagnetic radiation at 254 nm from the radiation-emitting device 114 may be controlled by the control circuitry 116 based on the detection of a living specimen as described.

In some embodiments, each time delay may be about 1 minute, about 2 minutes, about 3 minutes, about 4 minutes, about 5 minutes, about 10 minutes, greater than about 10 minutes, or individual values or ranges therebetween.

In some embodiments, the first time delay may be set to a time that indicates a low likelihood of the presence of a living specimen near the downlight apparatus 100. For example, where the control circuitry 116 does not detect a living specimen for a period of time, (i.e., the first time delay), the likelihood of the presence or imminent presence of a living specimen may be relatively low. Accordingly, the control circuitry 116 may be programmed to deactivate the visible light assemblies 120 after the first time delay.

In some embodiments, the second time delay may be equal to the first time delay. In some embodiments, the second time delay may be greater than the first time delay, thereby ensuring that the radiation-emitting device 114 may be safely activated to emit the first and second electromagnetic radiations.

In some embodiments, the third time delay may be based on the time required to emit a sanitizing dose of electromagnetic radiation to space. In some embodiments, the space may be an entire room and/or an intended area of coverage (e.g., a volume or a surface area) by the downlight apparatus (i.e., the downlight aperture 106). Factors such as the size of a room, the height of the downlight apparatus 100 in the room, and/or the intensity of radiation emitted by the radiation-emitting device 114 may affect the time required to emit a sanitizing dose of electromagnetic radiation. Accordingly, the third time delay may be based on an effective amount of time to emit the sanitizing dose. In some embodiments, the third time delay may be adjusted by a user. For example, the control circuitry 116 may receive input from a user through an input device (e.g., one or more buttons) to adjust or set the third time delay. The input may be related to a desired time delay, a size of the room in which the downlight apparatus 100 is situated, a size of the intended area of coverage, and/or the height of the downlight apparatus 100 in the room. Accordingly, the third time delay may be set based on the intended room or area of coverage.

The radiation-emitting device 114 may comprise any device operable to emit radiation within the above-described electromagnetic radiation ranges, including, but not limited to, light-emitting diodes (LEDs), mercury vapor discharge devices, laser diodes (LDs), pulsed xenon lasers, fiber lasers, additional types of lasers, and additional types of radiation sources as would be apparent to a person having an ordinary level of skill in the art.

In some embodiments, the radiation assembly 110 may further comprise a status-indicating device. In some embodiments, the status-indicating device may include one or more LEDs configured to emit light within the visible spectrum. In some embodiments, at least some of status-indicating device may be operable to emit light perceived as red, i.e. within a wavelength range from 625 nm to 740 nm when it is desired to indicate an error with respect to the specimen within the downlight aperture 106 or otherwise indicate the specimen should not pass through the downlight aperture 106. In some embodiments, the status-indicating device may be operable to emit light perceived as green, i.e. within a wavelength range from 500 nm to 565 nm, when it is desired to indicate that the specimen can proceed out of the downlight aperture 106. In some embodiments, the status-indicating device may be operable to emit light perceived as yellow, i.e. within a wavelength range from 565 nm to 590 nm, to indicate that the downlight aperture 106 is being irradiated with UV radiation. Additional and/or alternate status messages may be communicated through the use of the status-indicating device.

In some embodiments, the control circuitry 116 may include a motion sensor to detect the presence and/or location of an object within the downlight aperture 106. In embodiments in which the downlight apparatus 100 is installed in a structure, the motion sensor may be used not only to detect the presence of a moving object within a room or structure, but also the location of one or more persons within the room or structure. The radiation-emitting device 114 may then be configured to emit radiation based on a signal received from the motion sensor. Further, the radiation-emitting device 114 may also be configured to emit radiation in a specific direction within the room or structure. For example, if the motion sensor detects a new presence within the room or structure, e.g., a new guest or person who has entered the room or structure, then the motion sensor may send a signal to the radiation-emitting device 114 to cause radiation to be emitted only in the direction of the newly detected presence within the room or structure. Also, for example, if the motion sensor detects a new presence within the room or structure, but also detects that a different presence has not changed (i.e., one person has been in a room, but another person has entered a room), the motion sensor may send a signal to the radiation-emitting device to 114 to cause radiation to be emitted only in the direction of the newly sensed presence within the room or structure.

It is also contemplated that a user may manually operate the radiation-emitting device 114 as desired. For example, if a user is in an office where the downlight apparatus 100 is installed, and one or more individuals enter the office, the user may wish to operate the radiation-emitting device 114 upon entry of new guest in the office. In such a case, the user may also desire to select a location within the office to direct emission of the radiation from the radiation-emitting device 114. For example, if two guests enter the user's office and sit on opposite sides of a desk from the user, the user may operate the radiation-emitting device 114 to emit radiation only in the direction of the sides of the desk where the guests are located.

It is also contemplated that a user may manually operate the visible light assemblies 120. For example, the visible light assemblies may additionally or alternatively be controlled by a light switch, a wall switch, a remote control, or other types of control devices as would be known to a person having an ordinary level of skill in the art.

Additional information on downlight apparatuses may be found in U.S. patent application Ser. No. 16/108,225 titled Low Profile Light, the entire contents of which are incorporated herein by reference, as well as the contents of U.S. patent application Ser. No. 15/647,334 titled Low Profile Light, U.S. patent application Ser. No. 15/237,804 titled Low Profile Light and Accessory kit for the Same, U.S. patent application Ser. No. 14/492,348 titled Low Profile Light and Accessory kit for the Same, U.S. patent application Ser. No. 14/134,884, titled Low Profile Light and Accessory kit 2013, U.S. patent application Ser. No. 13/476,388, titled Low Profile Light and Accessory kit for the Same, U.S. patent application Ser. No. 12/775,310, Low Profile Light, and U.S. Provisional Application Ser. No. 61/248,665, titled Low Profile Light filed Oct. 5, 2009.

It is further contemplated and included within the scope of this disclosure that a similar radiation methodology may be employed in other devices. For example, a device situated within or replacing and/or retrofitting a dome light within a passenger vehicle may similarly be operable to emit UV radiation for deactivation of pathogens within the vehicle. Vehicles providing commercial passenger services, for example taxis, ridesharing vehicles, buses, shuttles, airlines, trains or ferries, may specifically benefit.

The present disclosure further contemplates that an existing downlight apparatus may be retrofitted to include the downlight apparatus disclosed herein.

Some of the illustrative aspects of the present disclosure may be advantageous in solving the problems herein described and other problems not discussed which are discoverable by a skilled artisan.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Instead, this application is intended to cover any variations, uses, or adaptations of the present teachings and use its general principles. Further, this application is intended to cover such departures from the present disclosure as come within known or customary practice in the art to which these teachings pertain. Many modifications and variations can be made to the particular embodiments described without departing from the spirit and scope of the present disclosure, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A downlight apparatus that utilizes UV radiation to deactivate pathogens comprising:
a housing;
a base member, wherein the base member is attached to the housing;
a light source configured to emit light in the visible spectrum; and
one or more ultraviolet radiation assemblies within the housing;
wherein the housing is configured to define a downlight aperture region irradiated by the light source and the one or more ultraviolet radiation assemblies.

2. The apparatus of claim 1, wherein the base member comprises an Edison base.

3. The apparatus of claim 1, wherein the one or more ultraviolet radiation assemblies are configured to be attached to the base member using one or more brackets.

4. The apparatus of claim 1, wherein the downlight aperture region is configured to encompass a human.

5. The apparatus of claim 1, further comprising a control circuit coupled to the one or more ultraviolet radiation assemblies.

6. The apparatus of claim 5, further comprising one or more sensors coupled to the control circuit.

7. The apparatus of claim 6, wherein at least one of the one or more sensors is configured to detect radiation either emitted by or reflected from an object within the downlight aperture region.

8. The apparatus of claim 7, wherein the detected radiation comprises infrared radiation from an individual occupying the downlight aperture region.

9. The apparatus of claim 7, wherein the detected radiation comprises reflected radiation generated by the one or more ultraviolet radiation assemblies.

10. The apparatus of claim 6, wherein at least one of the one more sensors is a motion sensor.

11. The apparatus of claim 6, wherein the control circuit, based on input from at least one of the one or more sensors, is configured to determine whether an object within the downlight aperture region is a living organism.

12. The apparatus of claim 1, wherein at least one of the one or more ultraviolet radiation assemblies is configured to emit electromagnetic radiation having a peak intensity within a range from 185 nm to 235 nm.

13. The apparatus of claim 12, wherein the at least one of the one or more ultraviolet radiation assemblies are configured to emit electromagnetic radiation having a peak intensity within a range from 217 nm to 227 nm.

14. The apparatus of claim 13, wherein the at least one of the one or more ultraviolet radiation assemblies are configured to emit electromagnetic radiation having a peak intensity of 222 nm.

15. The apparatus of claim 1, wherein at least one of the one or more ultraviolet radiation assemblies are configured to emit electromagnetic radiation having a peak intensity within a range from 249 nm to 259 nm.

16. The apparatus of claim 11, wherein, responsive to a determination that the object is a living organism, the control circuit is configured to:
activate the light source; and
deactivate at least one of the one or more ultraviolet radiation assemblies.

17. The apparatus of claim 11, wherein, responsive to a determination that the object is not a living organism, the control circuit is configured to:
deactivate the light source;
activate at least one of the one or more ultraviolet radiation assemblies to irradiate the downlight aperture region; and
deactivate the at least one of the one or more ultraviolet radiation assemblies after a preconfigured period of time.

18. The apparatus of claim 17, wherein at least one of deactivating the light source, activating at least one of the one or more ultraviolet radiation assemblies, and deactivating at least one of the one or more ultraviolet radiation assemblies is performed on a preconfigured time delay.

19. The apparatus of claim 11, wherein the control circuit is configured to:
activate the light source based on the input from the one or more sensors indicative of a presence of one or more living organisms within the portal aperture region;
deactivate the light source based on the input from the one or more sensors indicative of an absence of living organisms with the portal aperture region; and
selectively activate the one or more ultraviolet radiation assemblies according to a preconfigured schedule.

20. The apparatus of claim 1, wherein the one of the one or more ultraviolet radiation assemblies comprise one or more of a light-emitting diode, a laser diode, a pulsed xenon laser, and a fiber laser.

21. The apparatus of claim 1 further comprising a transparent or translucent cap attached to the housing, wherein light emitted by the light source passes through the transparent or translucent cap.
